(19) United States
(12) Patent Application Publication
(10) Pub. No.: US 2025/0290138 A1
(43) Pub. Date: Sep. 18, 2025

(54) METHOD FOR PREDICTION OF RECURRENCE OR PROGNOSIS OF DIABETIC FOOT ULCER BY USING SPECIFIC METHYLATION OF GENE

(71) Applicant: KOREA UNIVERSITY RESEARCH AND BUSINESS FOUNDATION, Seongbuk-Gu Seoul (KR)

(72) Inventors: Sik NAM GOONG, Seocho-gu Seoul (KR); Seung Kyu HAN, Seocho-gu Seoul (KR); Jeong An GIM, Gunpo-si Gyeonggi-do (KR); Hyun Jyung OH, Guro-gu Seoul (KR); Hyup Woo LEE, Guro-gu Seoul (KR)

(21) Appl. No.: 18/027,561

(22) PCT Filed: Sep. 17, 2021
(86) PCT No.: PCT/KR2021/012844

§ 371 Date: Mar. 21, 2023

(30) Foreign Application Priority Data
Sep. 21, 2020 (KR) 10-2020-0121179
Oct. 19, 2020 (KR) 10-2020-0135216

(51) Int. Cl. C12Q 1/6883; C12Q 1/686

(52) U.S. Cl. CPC C12Q 1/6883; C12Q 1/686; C12Q 2600/118; C12Q 2600/154; G01N 2800/54

(57) ABSTRACT

The present invention relates to a biomarker for predicting the reoccurrence or prognosis of diabetic foot ulcer, according to specific methylation of at least one gene selected from the group consisting of MORN1, NCOR2, and LINC00504. The present invention identifies a specific pattern of reoccurrence or prognosis of diabetic foot ulcer, discovers major biomarkers that account for the pattern, by machine learning of gene data, and thus, not only can predict the reoccurrence or prognosis of diabetic foot ulcer by using blood analysis-based clinical biomarkers, and but can also be used in the future to develop mechanisms for preventing or treating diabetic foot ulcer. By the discovery of factors that account for vital signs, the prediction of treatment or prognosis of diabetic foot ulcer is possible, and thus, a system capable of preemptively controlling disease progress can be established.

### CROSS-REFERENCE TO RELATED APPLICATIONS

This application is a U.S. National Phase Application of International Patent Application No. PCT/KR2021/012844, filed Sep. 17, 2021, which claims priority to Korean Patent Application No. 10-2020-0121179, filed Sep. 21, 2020, and which claims priority to Korean Patent Application No. 10-2020-0135216, filed Oct. 19, 2020, each of which is hereby incorporated by reference herein.

### REFERENCE TO AN ELECTRONIC SEQUENCE LISTING

The contents of the electronic sequence listing (Sequence Listing.txt; Size: 1,938 bytes; and Date of Creation: Aug. 18, 2023) is herein incorporated by reference in its entirety.

### Technical Field

The following description relates to a biomarker for predicting the recurrence of diabetic foot ulcer according to specific methylation of a gene, and the like.

### Background Art

Diabetes has a prevalence of more than 400 million people worldwide, and when diabetes occurs, the viscosity of the blood increases to gradually narrow or block the blood vessels. At this time, if the blood vessels going to the lower extremities are narrowed or blocked, the feet do not receive sufficient blood supply, and as a result, the supply of nutrients or oxygen through the blood is not smooth, and it is difficult to move and discharge wastes, thereby creating an environment where ulcers easily occur. Accordingly, about 15% to 25% of diabetic patients develop foot ulcer at a certain point in their lives, and since diabetes itself delays wound healing, once foot ulcer develops, self-healing is slow.

Meanwhile, diabetic neuropathy is a condition in which nerve damage caused by diabetes reduces sensation in the legs and feet. Even when an unhealed part due to compression or amputation occurs in the patient, the risk of ulceration increases due to decreased sensation in the legs and feet of the patient. Thereafter, if left untreated, the damaged portion expands rapidly and develops into foot ulcer susceptible to multimicrobial infection that causes tremendous tissue destruction.

Diabetic foot ulcer often recurs once suffered, and thus, about 30% of patients experience recurrence within 1 year, and more than half of patients who have surgery for diabetic foot ulcer will also have surgery on the opposite foot within 4 years after surgery. Excluding trauma, more than half of causes of partial leg amputation is diabetic foot ulcer, which is a very important cause of deteriorating the quality of life of diabetic patients.

It is known that when the diabetic foot ulcer occurs, the mortality rate after 5 years is about 43% to 55%, and when lower leg amputation occurs due to severe ulcer, the mortality rate within 5 years reaches 78%.

However, diabetic foot ulcer does not have a diagnostic code itself, has been recognized as only one of serious complications of diabetes, has no system for diagnosing underlying diabetic foot ulcer, and has no biomarker for diagnosing recurrence. Overseas, research on the treatment of diabetic foot ulcer as an incurable disease is mainly conducted, but research on searching of recurrence biomarkers and establishment of a recurrence prevention system is insufficient.

Unlike existing diabetes diagnostic biomarkers, biomarkers for diabetic foot ulcer differ from biomarkers for diabetic diagnosis because various evaluation of the degree of angiogenesis, infection, neuropathy, and cell healing ability needs to be comprehensively achieved.

Accordingly, the present inventors have studied specifically hypermethylated or hypomethylated genes according to the recurrence or prognosis of diabetic foot ulcer by using machine learning based on epigenomic data of patients with diabetic foot ulcer, and then completed the present disclosure.

### Disclosure of the Invention

### Technical Goals

An aspect of the present disclosure is to provide a biomarker for predicting the recurrence or prognosis of diabetic foot ulcer, including a CpG site of a gene.

Another aspect of the present disclosure is to provide a composition for predicting the recurrence or prognosis of diabetic foot ulcer including a preparation for measuring the methylation level of a CpG site of a gene, and a kit for predicting the recurrence or prognosis of diabetic foot ulcer including the same.

Yet another aspect of the present disclosure is to provide an information providing method or a diagnosis method for predicting the recurrence or prognosis of diabetic foot ulcer.

Still another aspect of the present disclosure is to provide a method for screening a biomarker for predicting the recurrence or prognosis of diabetic foot ulcer.

However, technical goals to be achieved are not limited to those described above, and other goals not mentioned above are clearly understood by one of ordinary skill in the art from the following description.

### Technical Solutions

In order to achieve the aspect of the present disclosure, an aspect of the present disclosure provides a biomarker for predicting the recurrence or prognosis of diabetic foot ulcer, including a CpG site of a gene.

As an embodiment of the present disclosure, the gene may be at least one selected from the group consisting of MORN1, NCOR2, and LINC00504.

As another embodiment of the present disclosure, the CpG site of the MORN1 gene may include a CpG site (cg01296877) shown in a nucleotide sequence (SEQ ID NO: 1) at positions 2274928 to 2275044, which is an intron region of Chromosome 1 based on human genome assembly hg19.

As yet another embodiment of the present disclosure, the CpG site of the NCOR2 gene may include a CpG site shown in a nucleotide sequence (SEQ ID NO: 2) at positions 124827121 to 124827421, which is an intron region of Chromosome 12 based on human genome assembly hg19, desirably a CpG site (cg23878260) shown at position 124827315 of Chromosome 12.

As still another embodiment of the present disclosure, the CpG site of the LINC00504 gene may include a CpG site (cg16033700) shown at position 14868419 of Chromosome 4 based on human genome assembly hg19.

Table 1 below shows SEQ ID NO: 1 (117 bp) included in the CpG site of the MORN1 gene and SEQ ID NO: 2 (301 bp) included in the CpG site of the NCOR2 gene before bisulfite treatment. When bisulfite is treated to measure the methylation level of the CpG site of the gene, C is changed to T.

TABLE 1

| | |
|---|---|
| SEQ ID NO: 1: | ATGCAGTGGAGGTGAGGAGAACCCTGAGGACAGCAGCCG |
| | CAGGGAGCCCCGGACCTTGGCCAGGCCCCCAGGCCCGAG |
| | TCTCAGTCCTCGAGGGGCCACTGCTGCTCAGCACAGCAC |
| SEQ ID NO: 2: | GGATTGGAAGACCCAACAGTGTAATCATGAGCTTAGGTTG |
| | GAGCAGAATTTCTCTTAGTAGTTTGCAGGACATGTGGGGT |
| | TAAACATTTCAGTGGTTTTCTTTTCCGGCAGGACTTATCAG |
| | TGCCTTTAGCAATGCAAAGGTATAGAATGAGGACTTGAGT |
| | ATATGCATTTTTCAAATAGACATGATCTGAAAGTCTTTTTTA |
| | AAAGTTGCCGGGCACGGTGGCTCACACCTGTAATCCCAGC |
| | ACTTTGGGAGGCCGAGGCAGGCGGATCACAAGGTCAGGA |
| | GATAGAGACCATCCTGGCT |

Another aspect of the present disclosure provides a composition for predicting the recurrence or prognosis of diabetic foot ulcer including a preparation for measuring the methylation level of a CpG site of a gene, and a kit for predicting the recurrence or prognosis of diabetic foot ulcer including the same.

As an embodiment of the present disclosure, the gene may be at least one selected from the group consisting of MORN1, NCOR2, and LINC00504.

As another embodiment of the present disclosure, the preparation for measuring the methylation level of the CpG site of the gene may include bisulfite or a methylation sensitive restriction enzyme, a primer specific to a methylated sequence of the CpG site of the gene, a primer specific to an unmethylated sequence, a methylated CpG binding domain, or an antibody specifically binding to methylcytosine.

As yet another embodiment of the present disclosure, the primer specific to the methylated sequence of the CpG site of the gene may be at least one selected from the group consisting of primers represented by SEQ ID NOs: 3 to 6.

TABLE 2

| | | |
|---|---|---|
| SEQ ID NO: 3: | MORN1 forward | ATGTAGTGGAGGTGAGGAGAATTT |
| | primer | |
| SEQ ID NO: 4 | MORN1 reverse | ATACTATACTAAACAACAATAACCCCT |
| | primer | |
| SEQ ID NO: 5 | NCOR2 forward | GGATTGGAAGATTTAATAGTGTAA |
| | primer | |
| SEQ ID NO: 6 | NCOR2 reverse | AACCAAAATAATCTCTATCTCCTAACC |
| | primer | T |

Yet another aspect of the present disclosure provides an information providing method or a diagnosis method for predicting the recurrence or prognosis of diabetic foot ulcer, the method including the steps of:
(a) isolating DNA from a clinical sample:
(b) measuring the methylation level of a CpG site of at least one gene selected from the group consisting of MORN1, NCOR2, and LINC00504 in the isolated DNA: and
(c) comparing the methylation level of the CpG site of the gene with the methylation level of a CpG site in the same genome region of a control group in which diabetic foot ulcer has not recurred.

As another embodiment of the present disclosure, after the comparing step, the method may include predicting that the risk of recurrence of diabetic foot ulcer is high when the CpG site of the MORN1 gene promoter is hypomethylated.

As yet another embodiment of the present disclosure, after the comparing step, the method may include predicting that the risk of recurrence of diabetic foot ulcer is high when the expression of the MORN1 gene is not inhibited compared to the control group in which diabetic foot ulcer has not recurred.

As still another embodiment of the present disclosure, after the comparing step, the method may include predicting that the risk of recurrence of diabetic foot ulcer is high when the CpG site of the NCOR2 gene promoter is hypermethylated.

As still yet another embodiment of the present disclosure, after the comparing step, the method may include predicting that the risk of recurrence of diabetic foot ulcer is high when the expression of the NCOR2 gene is further inhibited than the control group in which diabetic foot ulcer has not recurred.

As another embodiment of the present disclosure, after the comparing step, the method may include predicting that the risk of recurrence of diabetic foot ulcer is high when the CpG site of the LINC00504 gene promoter is hypermethylated.

As yet another embodiment of the present disclosure, after the comparing step, the method may include predicting that the risk of recurrence of diabetic foot ulcer is high when the expression of the LINC00504 gene is further inhibited than the control group in which diabetic foot ulcer has not recurred.

As yet another embodiment of the present disclosure, the clinical sample may be at least one selected from the group consisting of tissue, sputum, cells, blood, plasma and urine derived from a patient suspected of having the recurrence of diabetic foot ulcer.

As still another embodiment of the present disclosure, the measurement of the methylation level may be performed by at least one selected from the group consisting of polymerase chain reaction (PCR), methylation specific PCR, real time methylation specific PCR, PCR using methylated DNA specific binding protein, quantitative PCR, DNA chip, pyrosequencing, and bisulfite sequencing.

Yet another aspect of the present disclosure provides a use of at least one gene selected from the group consisting of MORN1, NCOR2, and LINC00504 for preparing a drug for diagnosing the recurrence or predicting the prognosis of diabetic foot ulcer.

Still another aspect of the present disclosure provides a method for screening a biomarker for predicting the recurrence or prognosis of diabetic foot ulcer, the method including the steps of:
(a) isolating DNA from a clinical sample:
(b) acquiring the methylation level of a CpG site of a gene from the isolated DNA: and
(c) screening genes associated with the recurrence of diabetic foot ulcer based on the methylation information using a decision tree.

Still yet another aspect of the present disclosure provides a composition for preventing or treating the recurrence of diabetic foot ulcer.

As an embodiment of the present disclosure, the composition may include at least one selected from the group consisting of a preparation for preventing hypomethylation of the CpG site of the MORN1 gene, a preparation for preventing hypermethylation of the CpG site of the NCOR2 gene, and a preparation for preventing hypermethylation of the CpG site of the LINC00504 gene.

Still yet another aspect of the present disclosure provides a method for preventing or treating the recurrence of diabetic foot ulcer.

As an embodiment of the present disclosure, the method may further include administering, to a subject, at least one preparation selected from the group consisting of a preparation for preventing hypomethylation of the CpG site of the MORN1 gene, a preparation for preventing hypermethylation of the CpG site of the NCOR2 gene, and a preparation for preventing hypermethylation of the CpG site of the LINC00504 gene.

### Effects

The present disclosure relates to a biomarker for predicting the recurrence or prognosis of diabetic foot ulcer, according to specific methylation of at least one gene selected from the group consisting of MORN1, NCOR2, and LINC00504. The present disclosure identifies a specific pattern of recurrence or prognosis of diabetic foot ulcer, discovers major biomarkers that account for the pattern, by machine learning of gene data, and thus, not only may predict the recurrence or prognosis of diabetic foot ulcer by using blood analysis-based clinical biomarkers, and but may also be used in the future to develop mechanisms for preventing or treating diabetic foot ulcer. By the discovery of factors that account for vital signs, the prediction of treatment or prognosis of diabetic foot ulcer is possible, and thus, a system capable of preemptively controlling disease progress may be established.

### BRIEF DESCRIPTION OF DRAWINGS

FIG. 1 is a Venn diagram illustrating hypermethylated genes according to recurrence and hypermethylated genes according to good or bad prognosis.

FIG. 2 illustrates each condition of epigenetic factors according to a recurrence condition as a decision tree. At this time, when a probe is a MORN1 gene and the methylation level of cg01296877 as a major node is 0.394 or less, it may be determined that the risk of recurrence of diabetic foot ulcer is high.

### Best Mode for Carrying Out the Invention

The present inventors have made an intensive study on the prediction of recurrence or prognosis of diabetic foot ulcer, and as a result, identified that the methylation levels of CpG sites of some genes vary according to the recurrence or prognosis of diabetic foot ulcer, and then completed the present disclosure.

More specifically, the present inventors determined that when the CpG site of the MORN1 gene was hypomethylated, when the CpG site of the NCOR2 gene was hypermethylated, and when the CpG site of the LINC00504 gene was hypermethylated, the risk of the recurrence of diabetic foot ulcer may be predicted.

Accordingly, the present disclosure may provide a biomarker for predicting the recurrence or prognosis of diabetic foot ulcer including a CpG site of a gene, a composition for predicting the recurrence or prognosis of diabetic foot ulcer including a preparation for measuring the methylation level of a CpG site of a gene and a kit including the same, an information providing method or a diagnosis method for predicting the recurrence or prognosis of diabetic foot ulcer, and a method for screening a biomarker for predicting the recurrence or prognosis of diabetic foot ulcer.

In the present disclosure, the term “the recurrence of diabetic foot ulcer” means a case where ulcer newly occurs in the same part of the same foot, in different parts of the same foot, or in a specific part of the opposite foot after existing diabetic foot ulcer was judged as complete healing or after at least 6 months have elapsed. The “complete healing of diabetic foot ulcer” refers to a condition in which a wound region of diabetic foot ulcer is completely epithelialized, and then may be showered because there is no open part or discharge.

As used herein, the term “methylation” means that a methyl group is attached to a base constituting DNA. Desirably, in the present disclosure, the methylation refers to whether methylation occurs in cytosine of a CpG site of a specific gene. When the methylation occurs, the binding of a transcription factor is disturbed to inhibit the expression of the specific gene. Conversely, when unmethylation or hypomethylation occurs, the expression of the specific gene is increased.

In genomic DNA of mammalian cells, in addition to A, C, G, and T, there is the fifth base called 5-methylcytosine (5-mC) in which a methyl group is attached to the fifth carbon of a cytosine ring. The methylation of 5-methylcytosine occurs only at C of CG dinucleotide (5′-mCG-3′) called CpG, and the methylation of CpG inhibits the expression of transposon and a repeating sequence of a genome. In addition, since the 5-mC of the CpG is naturally deaminated to be easily thymine (T), the CpG is a site where most epigenetic changes frequently occur in mammalian cells.

In the present disclosure, the term “measurement of the methylation level” refers to measuring the methylation level of the CpG site of the gene, and is performed by methylation specific PCR, such as methylation-specific polymerase chain reaction (MSP-PCR), real time methylation-specific PCR, PCR using methylated DNA-specific binding protein, or quantitative PCR. Alternatively, the methylation level may be measured by methods of automated sequencing such as pyrosequencing or bisulfite sequencing, or using a DNA methylation microarray, or immunoprecipitation using methylated CpG binding domains or anti-methylcytosine antibodies, but the present disclosure is not limited thereto.

In the present disclosure, the term “the CpG site of the gene” refers to a CpG site present on the DNA of the gene. The DNA of the gene is a concept including all of a series of structural units required for the expression of the gene and operably linked to each other, and may include, for example, a promoter region, an open reading frame (ORF) and a terminator region. Accordingly, the CpG site of the gene may present in the promoter region, the open reading frame (ORF) or the terminator region of the corresponding gene. Desirably, a CpG site where disease-specific hypermethylation occurs in a TNFRSF19 gene may be present in the promoter of the gene.

In the present disclosure, the preparation for measuring the methylation level of the CpG site may include a compound that modifies an unmethylated cytosine base or a methylation-sensitive restriction enzyme, a primer specific to a methylated allelic sequence of a gene, a primer specific to an unmethylated allelic sequence, a methylated CpG binding domain, a methylated DNA antibody that specifically binds to methylated DNA (for example, antibody that specifically binds to methylcytosine, or the like.

The compound that modifies the unmethylated cytosine base may be bisulfite or a salt thereof, but is not limited thereto, and may be desirably sodium bisulfite. The methylation of the bisulfite-modified DNA may be detected through various methods such as sequencing, methylation-specific PCR, or the like, and a method of detecting whether a gene is methylated by modifying an unmethylated cytosine residue using such bisulfite is well known in the art (e.g., WO 01/26536; US 2003/0148326A1).

In addition, the methylation-sensitive restriction enzyme may be a restriction enzyme capable of specifically detecting the methylation of the CpG site, and may be a restriction enzyme containing CG as a recognition site of the restriction enzyme. For example, the methylation-sensitive restriction enzyme includes SmaI, SacII, EagI, HpaII, MspI, BssHII, BstUI, NotI, and the like, but is not limited thereto. Depending on methylation or unmethylation at C of the restriction enzyme recognition site, cleavage by the restriction enzyme varies and may be detected through PCR or southern blot analysis. Methylation-sensitive restriction enzymes other than the restriction enzymes are well known in the art.

As a representative example of measuring the methylation level at a specific CpG site of a gene of a subject, the methylation level may be measured by obtaining genomic DNA from a patient's sample, treating the obtained DNA with a compound that modifies an unmethylated cytosine base or a methylation-sensitive restriction enzyme, amplifying the treated DNA by PCR using a primer, and confirming the presence or absence of the amplified result product.

Accordingly, the preparation of the present disclosure may include a primer specific to the methylated allele sequence of the gene and a primer specific to the unmethylated allele sequence. In the present disclosure, the term “primer” refers to a nucleic acid sequence having a short free 3-terminal hydroxyl group, and a short nucleic acid sequence capable of forming a base pair with a complementary template and serving as a starting point for copying a template strand. The primer may initiate DNA synthesis in the presence of a reagent for polymerization (i.e., DNA polymerase or reverse transcriptase) and four different nucleoside triphosphates in appropriate buffer solution and temperature. In addition, the primer is sense and antisense nucleic acids with a sequence of 7 to 50 nucleotides and may incorporate an additional feature without changing the basic properties of the primer that serves as an initiation site of DNA synthesis.

The primer of the present disclosure may be desirably designed according to a sequence of a specific CpG site to be analyzed for methylation, and may be a primer pair capable of specifically amplifying cytosine that is methylated, not modified by bisulfite, respectively, and a primer pair capable of specifically amplifying cytosine that is not methylated, but modified by bisulfite.

Meanwhile, the methylated DNA antibody refers to an antibody that specifically binds to a methylated base in DNA. Specifically, the methylated DNA antibody may include an antibody having a property of recognizing and binding to methylated cytosine in the DNA chain, such as an antibody against methylcytosine. In addition, among commercially available methylated DNA antibodies, the methylated DNA antibody may be an antibody capable of specifically recognizing and binding to DNA in a methylated state described herein.

The methylated DNA antibody may be prepared by a conventional method using a methylated base, methylated DNA, etc. as an antigen. For example, in order to prepare the methylcytosine antibody, the antibody is prepared using DNA containing 5-methylcytidine, 5-methylcytosine, or 5-methylcytosine as an antigen, and then specific binding to methylcytosine in DNA may be selected as an indicator.

In addition, in the case of measuring the methylation level using a methylated CpG binding domain (MBD) or a methylated DNA antibody, after immunoprecipitation of methylated DNA using these materials, a specific CpG site may be identified through southern blot, PCR, microarray, or sequencing. In addition, for immunological detection or quantification of the antibody, a substrate, an appropriate buffer solution, a chromogenic enzyme or fluorescent substance marker, a secondary antibody labeled with a chromogenic enzyme or fluorescent substance, and a chromogenic substrate may be used. As the substrate, a nitrocellulose membrane, a 96-well plate made of a polyvinyl resin, a 96-well plate made of a polystyrene resin, and a slide glass made of glass may be used. As the chromogenic enzyme, peroxidase, alkaline phosphatase, and the like may be used. As the fluorescent substance, FITC, RITC, etc. may be used. As the chromogenic substrate liquid, 2,2′-azino-bis-(3-ethylbenzothiazoline-6-sulfonic acid (ABTS), o-phenylenediamine (OPD), or tetramethyl benzidine (TMB) may be used, and other radioactive isotope labels, latex bead labels, colloid labels, biotin labels, etc. may be used, but the present disclosure is not limited thereto.

In the composition and the kit, in addition to the preparation, polymerase agarose, a buffer solution required for electrophoresis, and the like may be additionally included. In addition, the kit may be implemented in the form of a DNA methylation microarray.

In the present disclosure, the term “probe” refers to an oligonucleotide present on a microarray chip when performing an experiment using the microarray chip for DNA methylation analysis in the present disclosure, and refers to a gene on the chip that binds to DNA of the patient by expressing light when complementarily binding to DNA obtained from a specimen (blood sample).

More specifically, the probe is a nucleic acid strand that is partially or completely complementary to the DNA of the patient, and is an oligonucleotide capable of binding to the DNA of the patient in a base-specific manner. Desirably, the probe is an oligonucleotide that is completely complementary to the DNA of the patient. The probe includes not only nucleic acids, but also any conventionally known nucleic acid derivatives capable of complementary binding including a peptide nucleic acid.

In the present disclosure, the term “decision tree” is one of data mining analysis techniques and is a method of finding decision rules based on a tree structure. The decision tree is a powerful and widely used analytic technique for classifying or predicting a group of interest into several subgroups by diagramming decision rules. A general algorithm of the decision tree has different formation processes in a stopping rule, pruning, and the like. The rules used in the decision tree are as follows.
1. Separation criteria: Child nodes are determined by determining which predictor variable is used and how to be separated best distinguishes the distribution of a target variable, but the degree of discriminating the distribution of the target variable is measured using purity or other classification criteria.
2. Stop criteria: It means a rule for specifying that no further separation occurs and the current node becomes a terminal node.
3. Pruning: A decision tree with too many nodes is likely to have very large prediction errors when applied to new data. Therefore, it is preferred to select a decision tree having a sub-tree structure with an appropriate size as a final model by removing inappropriate nodes from the formed decision tree.

In the present disclosure, the term “node” means a classification criterion for presenting questions or correct answers in the decision tree. The first classification criterion, that is, the first question is called a root node, and the last classification criterion is called a terminal node, and the final classification result is presented.

The present disclosure may have various modifications and various examples and specific examples will be illustrated in the drawings and described in detail in the detailed description. However, the present disclosure is not limited to specific embodiments, and it should be understood that the present disclosure covers all the modifications, equivalents and replacements within the idea and technical scope of the present disclosure. In the interest of clarity, not all details of the relevant art are described in detail in the present specification in so much as such details are not necessary to obtain a complete understanding of the present disclosure.

### EXAMPLE 1. SUBJECT OF STUDY

18 patients hospitalized with diabetic foot ulcer at Korea University Guro Hospital (among first hospitalized patients without recurrence of diabetic foot ulcer, 6 patients with good prognosis, 3 patients with variable prognosis, 2 patients with poor prognosis, among patients with recurrence of diabetic foot ulcer, 6 patients with good prognosis and 1 patient with variable prognosis), 1 patient without pathological findings of diabetic foot ulcer, and 1 patient with diabetic hand ulcer were selected as subjects of the study. The patients were classified into recurrence of diabetic foot ulcer, prognosis condition after treatment, age, sex, white blood cell count (WBC), hemoglobin (Hb) concentration, C-reactive protein (CRP) level, and the like. In addition, the patients were classified into 7 patients in a diabetic foot ulcer recurrence group and 13 patients in a control group without recurrence of diabetic foot ulcer. A total of 48 specimens were obtained by collecting the blood from the patients 2 to 4 times during the treatment.

### EXAMPLE 2. DNA METHYLATION ANALYSIS

Genomic DNA (gDNA) was obtained from leukocytes obtained from the blood of the patients by using a DNeasy Blood & Tissue kit according to a protocol provided by a manufacturer QIAGEN. The extracted gDNA was quantified using NanoDrop, and integrity was confirmed through gel loading. DNA was treated with bisulfite using an EpiTect®: Fast DNA Bisulfite kit from QIAGEN.

For the extracted and bisulfite-treated DNA sample, the methylation levels of 862,873 CpG sites were measured using an EPIC 850k methylation chip. Specifically, the methylation level for each gene was measured using NGS or a microarray chip. At this time, the methylation level is represented by a = value of 0 to 1, and the β value of 0 means that the corresponding CpG site is completely unmethylated, and the β value of 1 means that the corresponding CpG site is completely methylated.

Preprocessing was performed to convert the measured DNA methylation level into a dataframe form used in Python or R, remove missing values, and connect patient information with clinical information of the patient. Thereafter, statistically significant results were derived through a T-test to identify differentially methylated genes in a patient group with recurrence of diabetic foot ulcer and differentially methylated genes in a patient group with good prognosis after treatment.

More specifically, the averages of the methylation levels (β values) of a patient group with recurrence of diabetic foot ulcer and a patient group without recurrence were obtained, respectively, and genes with CpG sites having a difference between the average values of 0.15 or more or −0.15 or less were selected. Among the genes, genes having a p-value<0.0005 or less using the T-test method were selected as a biomarker for predicting the recurrence or prognosis of diabetic foot ulcer. At this time, when the difference in methylation level was 0.15 or more, the gene was defined as hypermethylation, and when the difference was −0.15 or less, the gene was defined as hypomethylation. In the case of the patient group with good prognosis after treatment, the biomarkers were selected in the same manner as described above by using the patient group with poor prognosis after treatment as a control group.

### EXAMPLE 3. IDENTIFICATION OF SPECIFIC DNA METHYLATION CHANGES IN PATIENT GROUP WITH RECURRENCE OF DIABETIC FOOT ULCER

Probes showing specific DNA methylation changes in relation to recurrence and prognosis were selected, and visualized through a venn-diagram and a decision tree in relation to recurrence or prognosis.

More specifically, hypermethylated or hypomethylated probes were selected according to the recurrence or prognosis of diabetic foot ulcer (at this time, O in case of recurrence, X in case of non-recurrence, GD in case of good prognosis, and NG in case of poor prognosis were indicated, and in each case, hypermethylated genes were indicated as up.)

There was a total of 416 probes showing specific DNA methylation patterns related to recurrence or prognosis, in which included 200 probes related to recurrence, 225 probes related to prognosis, and 9 probes related to both recurrence and prognosis.

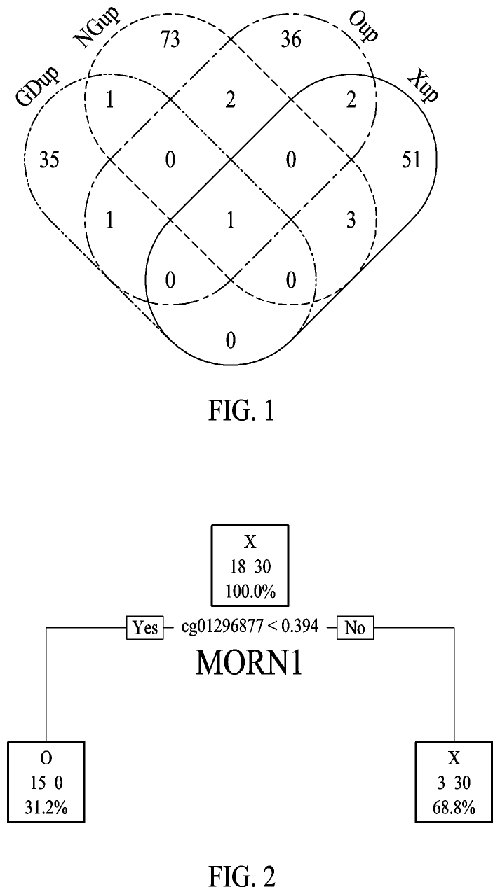

First, hypermethylated and hypomethylated genes according to recurrence and hypermethylated and hypomethylated genes according to good or bad prognosis were shown in a Venn diagram (FIG. 1). NCOR2 and LINC00504 genes were identified as commonly hypermethylated genes in samples with poor prognosis and recurrence. The results of analyzing DNA methylation changes of the two genes were as follows.

TABLE 3

| | | Difference in | | |
|---|---|---|---|---|
| | | methylation | | |
| | | level | Methylation | |
| Gene | Target ID | (β value) | state | p-value |
| NCOR2 | cg23878260 | 0.250000 | Hyper- | 0.0000416 |
| | | | methylated | |
| LINC00504 | Cg16033700 | 0.151801 | Hyper- | 0.000308 |
| | | | methylated | |

Each condition of epigenetic factors according to a recurrence condition was illustrated as a decision tree (FIG. 2). By using cg01296877 present in the MORN1 gene as a main node, when the methylation level of cg01296877 was lower than 0.394, it was classified as recurrence. More specifically, the methylation level of cg01296877 was lower than 0.394 in 15 samples among 18 samples in a group of diabetic foot ulcer recurrence. The remaining 3 samples and 30 samples in the group without recurrence showed the methylation levels of higher than 0.394. That is, when the methylation level of cg01296877 was lower than 0.394, there was a strong tendency to be classified as the high risk of recurrence of diabetic foot ulcer. The results of analyzing DNA methylation changes of the gene were as follows.

TABLE 4

| | | Difference in | | |
|---|---|---|---|---|
| | | methylation | | |
| | | level | Methylation | |
| Gene | Target ID | (β value) | state | p-value |
| MORN1 | cg01296877 | −0.384 | Hypo- | 0.00000000424 |
| | | | methylated | |

According to the results, it was confirmed that when the MORN1 gene was hypomethylated, when the NCOR2 gene was hypermethylated, or when the LINC00504 gene was hypermethylated as a non-coding gene, the risk of recurrence of diabetic foot ulcer was high. This indicated that the NCOR2, LINC00504, and MORN1 genes were effective as biomarkers for accurately determining the patient group with recurrence of diabetic foot ulcer compared to the control group without recurrence of diabetic foot ulcer.

As described above, specific parts of the present disclosure have been described in detail, and it will be apparent to those skilled in the art that these specific techniques are merely preferred embodiments, and the scope of the present disclosure is not limited thereto. Therefore, the substantial scope of the present disclosure will be defined by the appended claims and their equivalents.

### Industrial Applicability

The present disclosure relates to a biomarker for predicting the recurrence or prognosis of diabetic foot ulcer, according to specific methylation of at least one gene selected from the group consisting of MORN1, NCOR2, and LINC00504. The present disclosure not only may predict the recurrence or prognosis of diabetic foot ulcer with high recurrence rate and mortality using the biomarkers, but also may be used to develop a preventive or therapeutic mechanism for diabetic foot ulcer in the future. In addition, the present disclosure may establish a system capable of preemptively controlling disease progress by discovering factors capable of accounting for vital signs, and thus, may be usefully used in prevention, treatment and prognosis fields of diabetic foot ulcer.

### SEQUENCE LISTING FREE TEXT

Sequence listing 1

1. A biomarker composition for predicting the recurrence of diabetic foot ulcer, comprising a CpG site of a gene,
wherein the gene is at least one selected from the group consisting of MORN1, NCOR2, and LINC00504.

2. The composition of claim 1, wherein the CpG site of the MORN1 gene comprises CpG shown in a nucleotide sequence of SEQ ID NO: 1 (at positions 2274928 to 2275044 of Chromosome 1 based on assembly hg19).

3. The composition of claim 1, wherein the CpG site of the NCOR2 gene comprises CpG shown in a nucleotide sequence of SEQ ID NO: 2 (at positions 124827121 to 124821421 of Chromosome 12 based on assembly hg19).

4. The composition of claim 1, wherein the CpG site of the LINC00504 gene comprises CpG located in an intron of Chromosome 4.

5. A composition for predicting the recurrence of diabetic foot ulcer comprising a preparation for measuring the methylation level of a CpG site of a gene,
wherein the gene is at least one selected from the group consisting of MORN1, NCOR2, and LINC00504.

6. The composition of claim 5, wherein the preparation for measuring the methylation level of the CpG site of the gene comprises bisulfite or its salt, a methylation sensitive restriction enzyme, a primer specific to a methylated sequence of the CpG site of the gene, a primer specific to an unmethylated sequence, a methylated CpG binding domain, or an antibody specifically binding to methylcytosine.

7. The composition of claim 6, wherein the primer specific to the methylated sequence of the CpG site of the gene is at least one selected from the group consisting of primers represented by SEQ ID NOs: 3 to 6.

8. A kit for predicting the recurrence of diabetic foot ulcer comprising the composition of claim 5.

9. An information providing method for predicting the recurrence of diabetic foot ulcer comprising:
(a) isolating DNA from a clinical sample;
(b) measuring the methylation level of a CpG site of at least one gene selected from the group consisting of MORN1, NCOR2, and LINC00504 in the isolated DNA; and
(c) comparing the methylation level of the CpG site of the gene with the methylation level of a CpG site in the same genome region of a control group in which diabetic foot ulcer has not recurred.

10. The information providing method of claim 9, comprising:
after the comparing, predicting that the risk of recurrence of diabetic foot ulcer is high when the CpG site of the MORN1 gene is hypomethylated.

11. The information providing method of claim 9, comprising:
after the comparing, predicting that the risk of recurrence of diabetic foot ulcer is high when the CpG site of the NCOR2 gene is hypermethylated.

12. The information providing method of claim 9, comprising:
after the comparing, predicting that the risk of recurrence of diabetic foot ulcer is high when the CpG site of the LINC00504 gene is hypermethylated.

13. The information providing method of claim 9, wherein the clinical sample is at least one selected from the group consisting of tissue, sputum, cells, blood, plasma and urine derived from a patient suspected of having the recurrence of diabetic foot ulcer.

14. The information providing method of claim 9, wherein the measurement of the methylation level is performed by at least one selected from the group consisting of polymerase chain reaction (PCR), methylation specific PCR, real time methylation specific PCR, PCR using methylated DNA specific binding protein, quantitative PCR, DNA chip, pyrosequencing, and bisulfite sequencing.

15. A method for diagnosing the recurrence of diabetic foot ulcer comprising:
(a) isolating DNA from a clinical sample;
(b) measuring the methylation level of a CpG site of at least one gene selected from the group consisting of MORN1, NCOR2, and LINC00504 in the isolated DNA; and
(c) comparing the methylation level of the CpG site of the gene with the methylation level of a CpG site in the same genome region of a control group in which diabetic foot ulcer has not recurred.

16. A method for screening a biomarker for predicting the recurrence of diabetic foot ulcer comprising:
(a) isolating DNA from a clinical sample;
(b) acquiring the methylation level of a CpG site of a gene from the isolated DNA; and
(c) screening genes associated with the recurrence of diabetic foot ulcer based on the methylation information using a decision tree.


SEQUENCE LISTING (6 sequences)

SEQ ID NO: 1 - DNA, 117 bp, Homo sapiens
atgcagtggaggtgaggagaaccctgaggacagcagccgcagggagccccggaccttggccaggcccccaggcccgagtctcagtcctcgaggggccactgctgctcagcacagcac

SEQ ID NO: 2 - DNA, 301 bp, Homo sapiens
ggattggaagacccaacagtgtaatcatgagcttaggttggagcagaatttctcttagtagtttgcaggacatgtggggttaaacatttcagtggttttcttttccggcaggacttatcagtgcctttagcaatgcaaaggtatagaatgaggacttgagtatatgcatttttcaaatagacatgatctgaaagtcttttttaaaagttgccgggcacggtggctcacacctgtaatcccagcactttgggaggccgaggcaggcggatcacaaggtcaggagatagagaccatcctggct

SEQ ID NO: 3 - DNA, 24 bp, Artificial Sequence
  MORN1 forward primer
atgtagtggaggtgaggagaattt

SEQ ID NO: 4 - DNA, 27 bp, Artificial Sequence
  MORN1 reverse primer
atactatactaaacaacaataacccct

SEQ ID NO: 5 - DNA, 24 bp, Artificial Sequence
  NCOR2 forward primer
ggattggaagatttaatagtgtaa

SEQ ID NO: 6 - DNA, 24 bp, Artificial Sequence
  NCOR2 reverse primer
atgtagtggaggtgaggagaattt